(19) United States
(12) Patent Application Publication
(10) Pub. No.: US 2016/0040248 A1
(43) Pub. Date: Feb. 11, 2016

(54) METHOD TO IMPROVE EXPRESSION AND OTHER BIOLOGICAL ANALYSIS

(71) Applicant: GENEKEY CORPORATION, Palo Alto, CA (US)

(72) Inventor: Raphael LEHRER, Palo Alto, CA (US)

(73) Assignee: Genekey Corporation, Palo Alto, CA (US)

(21) Appl. No.: 14/776,621

(22) PCT Filed: Mar. 14, 2014
(86) PCT No.: PCT/US14/29283

§ 371 Date: Sep. 14, 2015

Related U.S. Application Data
(60) Provisional application No. 61/785,190, filed on Mar. 14, 2013.

(51) Int. Cl. C12Q 1/68

(52) U.S. Cl. CPC C12Q 1/6886; C12Q 2600/158

(57) ABSTRACT

A method to verify single data points by substituting multiple data points that predict the original value measured is described. This is applied to determining gene expression levels.

### CROSS REFERENCE TO RELATED APPLICATION

This application claims priority from U.S. Ser. No. 61/785,190 filed Mar. 14, 2013. The content of that document is incorporated herein by reference.

### TECHNICAL FIELD

The invention is in the field of methods to reduce noise associated with genomic measurements and pathway analysis. It is exemplified by applying these methods to gene expression.

### BACKGROUND ART

There are many instances of employing assays to measure the level or quantity of a particular property or characteristic. Generally, one directly measures this level or quantity as a single data point. For example, in measuring expression of a particular gene, the level of mRNA corresponding to the encoded protein may be measured or the level of the protein itself may be assessed. Especially in complex systems, various factors contribute noise to cast doubt on the validity of such an individual measurement. For example, in measuring mRNA as an index of gene expression, one may be misled by the instability of the mRNA itself or by its rapid turnover into protein. Thus, a gene that is highly expressed may show low levels of mRNA because the original high levels of mRNA are rapidly degraded.

One key limitation of such technologies is the imperfect accuracy of measurement. Such “noise” is particularly prevalent in measurements of mRNA via microarrays, but is present in other measurement technologies as well. This noise can increase the challenge of performing analyses relying either on single markers or panels of markers, gene signatures, or pathway analysis.

One method to reduce noise is to average a large number of samples. However, this can obscure disease related differences, and in the case of personalized medicine, may not be possible. It would be useful to have a method to reduce the noise associated with such measurements. When a large number of data points are measured, e.g., full genome data, this becomes possible as interrelated variables also affect the value of the parameter of interest. In the case of gene expression, for example, the levels of active transcription factors that control expression of a gene of interest will influence the level of expression, and the level of the transcription factors can be predicted by assessing a multiplicity of measurements relating to genes controlled at least in part by the same transcription factors. Thus, the invention provides a way to assess the validity of a single measurement or providing a more accurate value thereof by evaluating it against a predicted value based on a multiparameter assessment system.

Further, pathway analysis methodologies suffer from the ability to focus only on one pathway at once. Since these pathways are interconnected, such analysis can be flawed as they do not take into account most of the interactions of the genes with the others. With the method of the invention, a portion of the influence of other pathways may be “encoded” into the single predicted measurement.

### DISCLOSURE OF THE INVENTION

The invention provides a method to reduce noise otherwise associated with a single measurement, specifically the level of expression of a gene or multiplicity of genes of interest.

In one aspect, the invention is directed to a method to assess the probability of the validity of a direct measurement, in a biological sample, of the expression level of a first gene encoding a protein which method comprises

measuring in said sample levels of expression of a multiplicity of additional genes encoding proteins wherein the levels of expression of said additional genes are controlled at least in part by at least one transcription factor that controls the level of expression of said first gene;

predicting the level of activity of the at least one transcription factor based on the levels of expression of said additional genes; and

predicting the level of expression of said first gene based on the predicted level of the at least one transcription factor;

wherein agreement of the level of expression of said first gene as measured and the level of expression of said first gene as predicted indicates the direct measurement of said level has a high probability of validity and wherein a disagreement between said levels indicates the direct measurement is less probable to be valid.

In many embodiments, more than one transcription factor is considered.

The general method of obtaining a predicted value based on a multiparameter system can also be applied simply to obtain a predicted value, without the necessity for comparing it to a directly measured value.

In another aspect, the method described above can be expanded to apply it to more than one gene, even to the entire complement of genes in the genome. The results obtained by comparing the various genes that were subjected to the method of the invention will permit an assessment of the relative weight of each transcription factor associated with the expression of a gene of interest. This permits refining the data for each individual gene based on the newly acquired knowledge of the relative influence of the various transcription factors that effect its expression.

Thus, the invention is directed to a method which comprises calculating the predicted value of a single measurement of expression based on a multiplicity of measurements of factors that influence the value of that single measurement. This method may be used to obtain the value associated with expression of a particular gene, or multiplicity of genes per se—i.e., without reference to direct measurement. In addition, by comparing the predicted value with the actual single measurement value, the validity of the results of the single measurement can be ascertained.

The methods of the invention are particularly relevant in expression analysis, since there is substantial noise associated with the original measurement(s). It is well known that conclusions that are drawn from the combination of numerous data points, each of which are noisy, provides a more accurate measurement than using a single data point of comparable noise. This invention uses mechanistic relationships between genes derived from literature curation to enhance the validity of measurement of expression.

### BRIEF DESCRIPTION OF THE DRAWINGS

FIG. 1 shows an exemplary known pathway that may be relevant to malignancy. In this figure, the various components of the pathway that are upregulated, downregulated, or not affected in a sample obtained from an individual is presented. The upregulation, downregulation or non-effect was ascertained by assessing the level of mRNA associated with each of the proteins in this pathway.

FIG. 2 shows the same pathway as in FIG. 1, analyzed in a sample from the same individual, but employing the method of the invention to determine upregulation, downregulation or non-regulation of the expression of the gene for the relevant proteins. The results obtained by using the method of the invention—i.e., basing the level of expression on a survey of relevant transcription factors, provides a different result as shown.

### MODES OF CARRYING OUT THE INVENTION

The invention is based on the concept that the measurement of a multiplicity of factors that influence the value of an individual parameter of interest as a basis for predicting the value of that parameter will yield a more accurate assessment of that individual parameter than will a single direct measurement of the parameter itself. In an abstract example, if the value of A is directly measured to provide a quantitative result, this value may be subject to error due to background noise and general inaccuracies associated with making just a single measurement. If the actual value of A is increased when B is increased, decreased when C is decreased, and enhanced when the values of D and E are increased, and if the way in which the values of B, C, D and E affect the value of A is understood, then measuring this collection of factors will allow calculation of a predicted value for A. This predicted value has a higher probability of being accurate than the measurement of A itself because results of four measurements have a higher probability of collective accuracy than the value of one measurement.

In reality, the values of B, C, D, and E may be noisy as well, and their relationships with A may have inaccuracies. A naïve application of the methods described in the above paragraph may therefore be problematic. The invention method can overcome this problem.

The “validity” of a result is meant to refer to whether the result accurately reflects the actual value of the parameter measured. That is, the expression level of a gene may refer to the actual level of mRNA generated. A direct measurement of this level will likely yield less accurate results than a predicted value of this level according to the method of the invention. The invention method thus can either serve to validate or invalidate a direct measurement of mRNA level or other measure of expression, or may itself be used to predict this value without the necessity for direct measurement. By “invalidate” simply means that the value obtained by the predictive value is different from that from the direct measurement. It is understood, of course, that these statements refer to probabilities, not certainties; and it is simply more probable that the predicted value found by the method of the invention will be more accurate and therefore more valid than a direct measurement.

One can calibrate the level of importance of each interrelated variable to the measurement of expression of a gene of interest via use of a database of past measurements related to genes whose expression is controlled by the same transcription factor as the gene of interest. For example, the degree to which each transcription factor influences a gene can be assessed by determining the degree to which activation of a given transcription factor predicts expression of the gene across a large database of relevant samples. By use of such calibration, a “predicted measurement” can be assigned to each gene to replace the measured value. Such predicted measurement will have less noise than the original measurement as it is based on hundreds of measurements rather than a single one.

In effect, the invention method relies on measuring a multiplicity of expression levels, using these to predict activity of known transcription factors, and then applying known interactions of these transcription factors with to provide a predicted value for the individual gene of interest. Thus, the invention provides a method to assess the validity of an individual measurement, and also to provide a more probable value of such a measurement. In addition to applying this methodology as a method to validate or invalidate direct measurement, in a biological sample, of an expression level of the first gene encoding a protein, the invention also provides simply a method to determine in a biological sample the expression level of a gene of interest. This method can be extended beyond transcription factors as well as to other measurement technologies such as proteomics.

When applied in this context, the sample is typically a biological fluid such as blood, serum, urine, semen, saliva and cerebrospinal fluid, or a sample of biopsied tissue. However, the sample could also be derived from plant cells, microorganism cells, or any system wherein indicators of gene expression are found. The organism from which the sample can be derived is any organism or single cell, including cells in cell culture, microorganisms, plants, animals of all kinds and humans.

The method of the invention can also be expanded to improve its accuracy by predicting values for more than one gene—two, three, 10, 100 or all the genes in the genome. (Thus, the range of genes used in this improvement is two—the total number in the genome. When a range such as this is provided, all internal integer values are included. Thus, a range of 2-10,000 would include a range of 5-10 or 7-250. This comment is made in order to avoid laborious recitations of ranges.)

In this improvement, by predicting the values for expression of multiple genes, the influence of the various transcription factors can be evaluated and weighted. This permits further refinement of the predicted value for any individual gene. Thus, in more detail, the method of the invention which includes use of weighting factors could be outlined as follows:

First, the relevant transcription factors that are applicable to the gene of interest or to as many genes as desired to be evaluated for expression, are determined from curation of available sources—the open literature, patent literature, databases, etc.

In addition to this evaluation of the relevant data, a database is obtained where data have been generated on a large number of biological samples. For example, Gene Logic's BioExpress system is a database of 22,000 human tissue samples with data generated on the Aggy HU133 2+platform. The data may be selected as those pertaining to the most relevant parameters—typically in samples that are comparable to those in which the gene of interest is to be evaluated. An analysis is done for the gene of interest that correlates the expression of the gene (as measured) with the activity level of the relevant transcription factors across the entire database. A regression model may be used. If there are known nonlinear dynamics associated with the interactions these can be built into the regression model or a linear model may be used. This results in weighting factors that can be used to quantitate the impact of the relevant transcription factors (TF) on the gene of interest. However, if weighting factors for each TF are not to be used, a natural default is to count each one equally.

In an alternative way to create weighting factors is to compare the power of each factor by its success. If more of the targets of a first TF are increased than of the targets of a second TF, then the first TF is given more weight than the second. This analysis can also be used as a proxy for the degree to which a TF is activated in a specific sample. A combination of these results may also be used.

Thus, at least three features of the invention method are relevant: a) the double-step approach to reducing the noise in the measurement of TF's and then using that to reduce the noise of the directly measured parameter in question; b) the fact that this crosses data types—although the expression level of the TF's is measured, it is their activation (e.g., phosphorylation state) that is predicted and then is used to predict the expression levels of their target genes; and c) the fact that this “noise reduction” approach is based on mechanistic data from the literature rather than purely analytic data.

The double-step becomes triple-step when using existing databases to determine the appropriate weights of the interaction, and because of c), above, the resultant “predicted” data is not just a lower noise version of the original, but also encodes its biological interactions. The expression of each gene is used to assess the activation of a pathway, which suffers from the fact that one is looking at a single pathway at a time, but this is ameliorated by encoding the biological interactions of each gene with other pathways directly into the data itself. If one is using the gene as an individual marker because, for example, expression of gene X is known to lead to disease Y, utilizing the predicted data rather than the original data turns this into a mechanistic assessment rather than purely correlative.

Because the interactions of the TF's with their targets are taken from the literature, the weights of those interactions need to be established, which is done by use of a database of other gene expression samples and tuning the weights according to the relationships across them. Although this is correlative, it doesn't suffer from the same problems as pure correlative analysis because the choice of transcription factors is based on measured values.

The following example is intended to illustrate but not to limit the invention.

### Example 1

### Determination of a Dysregulated Pathway

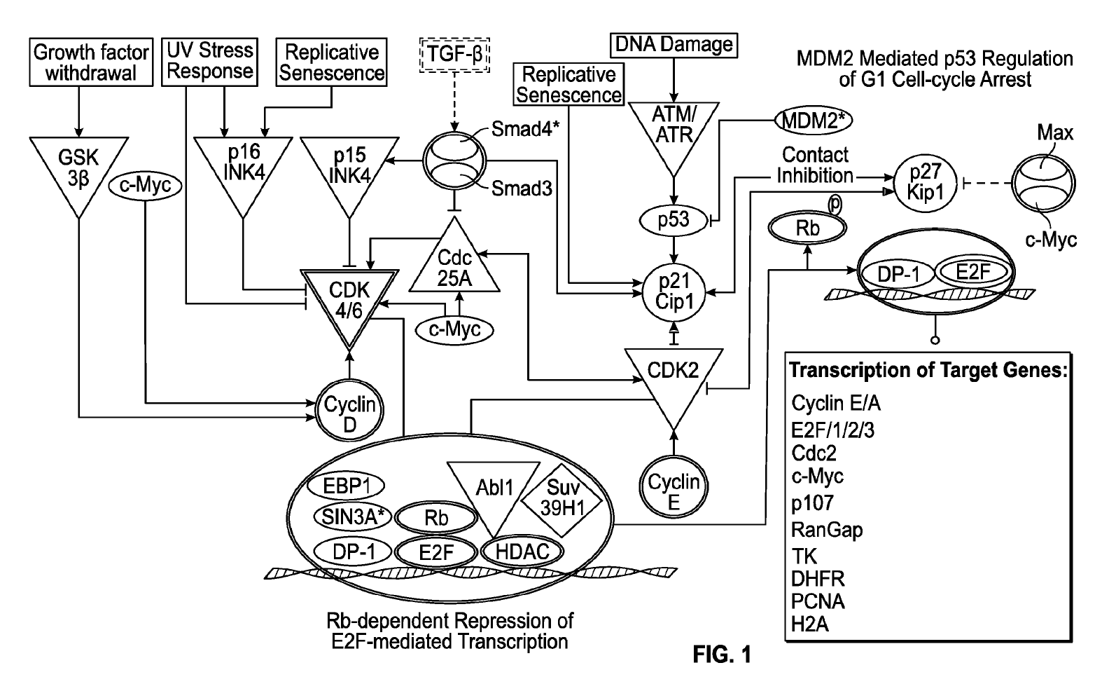

A colon cancer patient was evaluated to determine a disrupted pathway that might be a target for therapy by measuring the mRNA associated with a multiplicity of genes and matching the levels to known pathways mediated by these gene products. Using this approach, the status of the pathway shown in FIG. 1 was assessed. According to these results, the transcription factors c-Myc was not upregulated and E2F was also not appreciably upregulated.

To reassess the status of this pathway, sets of genes known to be regulated by the same transcription factor (TF) were measured. The status of these gene expression levels was then used to calculate the putative activity of the TF's that they share. This is done for multiple TF's. As examples, c-Myc and E2F were both predicted to be activated.

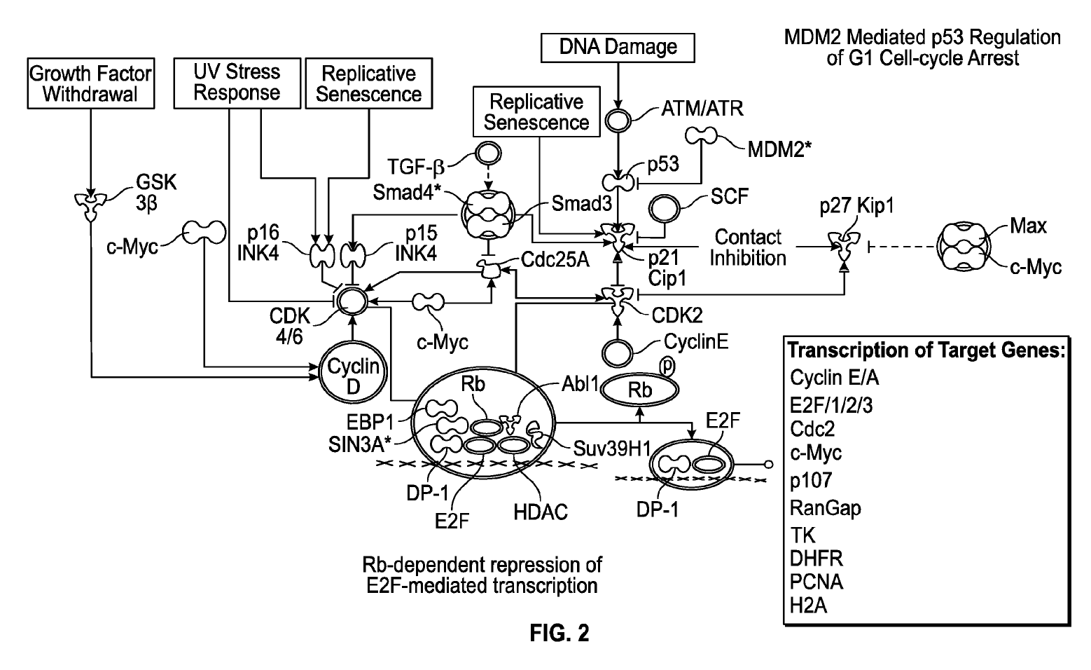

This analysis provides a multiplicity of data points each representing the activity of a TF based on the population of genes regulated by that TF. While each TF is involved in regulation of a multiplicity of genes, it is also true that each gene is regulated by a multiplicity of TF's. An algorithm was created that converts the activation state of each TF regulating a particular gene into a predicted effect on the gene. Based on the algorithm which involves, in this case, a total of 54 TF's, with an average of 5 TF's per individual gene, the pathway in FIG. 1 was reevaluated based on the conclusion of upregulation, downregulation or no prediction of each component to arrive at a revised status of this pathway in FIG. 2.

The pathway in FIG. 2, which indeed turned out to be a successful therapeutic target would have been rejected had the results of FIG. 1 been used to evaluate it. This is because upregulation or downregulation of 29 genes would have been predicted and the actual results showed that, of the 29, 12 were consistent with the involvement of the pathway in the disease, 8 were inconsistent and 9 were not significantly regulated. On the other hand, as shown in FIG. 2, of the 29, 20 were consistent with its involvement, 4 were inconsistent and 5 were neither.

In more detail, in FIG. 1, CDK6 is downregulated and CDK2 and CDK4 are unregulated which would be inconsistent with the involvement of this pathway. In FIG. 2, CDK2 and CDK4 are upregulated, which is consistent with its involvement.

Supplementary genes which changed from inconsistent to consistent with the hypothesis include Cyclin D, Cyclin E and p16NK4.

For completeness, the 54 transcription factors considered in this example were as follows:

| | | | |
|---|---|---|---|
| ATF4 | HOXB13 | NfkB1-RelA | RNF2 |
| CARM1 | JUN | NKX2-1 | SIM1 |
| CDX2 | KDM5B | NRF1 | SMARCA4 |
| CIITA | KLF2 | PAX3 | SOX2 |
| CREBBP | KLF6 | PGR | SOX9 |
| FOS | LEF1 | PIAS2 | SP1 |
| FOSB | MAF | PIAS3 | SRSF2 |
| FOXO1 | MAX | PPARA | Stat3-Stat3 |
| FOXO3 | MEOX2 | PPARD | TCF3 |
| GATA1 | NCOA2 | PPARG | TFAP2A |
| GATA6 | NCOR2 | PRDM1 | TOB1 |
| GLI2 | NEUROD1 | PROX1 | ZBTB17 |
| Hdac | NFE2L2 | PURA | |
| HMGA1 | NFIX | RARA | |

1. A method to validate or invalidate a direct measurement, in a biological sample, of the expression level of a first gene encoding a protein which method comprises
measuring in said sample levels of expression of a multiplicity of additional genes encoding proteins wherein the levels of expression of said additional genes are controlled at least in part by at least one transcription factor that controls the level of expression of said first gene;
predicting the level of activity of the at least one transcription factor based on the levels of expression of said additional genes;
predicting the level of expression of said first gene based on the predicted level of the at least one transcription factor;
wherein agreement of the level of expression of said first gene as measured and the level of expression of said first gene as predicted indicates the direct measurement of said level is valid and wherein a disagreement between said levels indicates the direct measurement is invalid.

2. The method of claim 1 wherein the levels of activity of at least two transcription factors that control the level of expression of the first gene are predicted.

3. The method of claim 2 wherein the levels of activity of at least five transcription factors that control the level of expression of the first gene are predicted.

4. A method to predict the level of gene expression of a first gene which method comprises
measuring in said sample levels of expression of a multiplicity of additional genes encoding proteins wherein the levels of expression of said additional genes are controlled at least in part by at least one transcription factor that controls the level of expression of said first gene;
predicting the level of activity of the at least one transcription factor based on the levels of expression of said additional genes; and
predicting the level of expression of said first gene based on the predicted level of the at least one transcription factor.

5. The method of claim 4 wherein the levels of activity of at least two transcription factors that control the level of expression of the first gene are predicted.

6. The method of claim 5 wherein the levels of activity of at least five transcription factors that control the level of expression of the first gene are predicted.

7. The method of claim 4 wherein said predicted level of expression is modified by applying an algorithm to the results of applying the method to a multiplicity of genes.

8. The method of claim 7 wherein the multiplicity of genes is at least 5.
